(19) United States
(12) Patent Application Publication
(10) Pub. No.: US 2026/0337665 A1
(43) Pub. Date: Oct. 29, 2026

(54) METHYLATION CONVERSION ASSESSMENT AND CONTROLS

(71) Applicant: TEMPUS AI, INC., Chicago, IL (US)

(72) Inventors: Nathaniel Perry Bailey, Winnetka, IL (US); Johathan Foox, New York, NY (US); Eric Leon Harness, Park Ridge, IL (US); Kevin Joseph Walsh, Chicago, IL (US)

(21) Appl. No.: 19/648,142

(22) Filed: Apr. 15, 2026

Related U.S. Application Data
(60) Provisional application No. 63/789,894, filed on Apr. 16, 2025.

(51) Int. Cl. C12Q 1/6827; C12Q 1/686; C12Q 1/6869

(52) U.S. Cl. CPC C12Q 1/6827; C12Q 1/686; C12Q 1/6869

(57) ABSTRACT

Provided herein is technology relating compositions and methods for analysis of methylated oligonucleotides from a subject. The disclosure provides a composition comprising four or more unique oligonucleotides, each of the four or more oligonucleotides comprising a unique nucleotide sequence and a unique number of methylated bases, wherein the unique number of methylated bases is about 0% to 100% of the unique nucleotide sequence of each of the four or more oligonucleotides, and uses thereof.

### CROSS REFERENCE TO RELATED APPLICATIONS

The present application claims the benefit of priority to U.S. Provisional Application No. 63/789,894, filed Apr. 16, 2025, the disclosure of which is incorporated herein by reference in its entirety.

### FIELD OF THE INVENTION

The disclosure provides a composition and a method for the analysis and quantification of oligonucleotide methylation in a sample.

### BACKGROUND

Methylated DNA has been studied as a potential class of biomarkers in the tissues of most tumor types. In many instances, DNA methyltransferases add a methyl group to DNA at cytosine-phosphate-guanine (CpG) island sites as an epigenetic control of gene expression. In a biologically active mechanism, acquired methylation events in promoter regions of tumor suppressor genes are thought to silence expression, thus contributing to oncogenesis. DNA methylation may be a more chemically and biologically stable diagnostic tool than RNA or protein expression (Laird (2010), Nat Rev Genet 11:191-203). Furthermore, in other cancers like sporadic colon cancer, methylation markers offer excellent specificity and may be more broadly informative and sensitive than individual DNA mutations (Zou et al (2007), Cancer Epidemiol Biomarkers Prev 16:2686-96).

Nucleic acids from patient samples, e.g., blood, stool, and tissue samples, that are analyzed for the presence of mutations and/or for methylation status associated with disease or risk of disease typically pass through a number of process steps during analysis. These steps may comprise, e.g., filtration, precipitation, capture, washing, elution, and/or chemical modification. For analysis of DNAs to determine methylation status, e.g., the percent methylation (or degree of methylation of DNA derived from a patient sample, processing typically comprises treatment with bisulfite to convert un-methylated dC bases to dU residues, making them more readily distinguishable from the methyl-C residues that are protected from bisulfite conversion. Examples of methylation degrees may include what percentage of DNA molecules have methylation at a specific methylation site, what percentage of methylation sites in a genomic region are methylated, what pattern of methylation sites in a genomic region are methylated (for example: x number of methylated sites, next to y number of unmethylated sites, next to z number of methylated sites, and so on throughout the region), or another metric of degree of methylation.

Accurate quantitation of oligonucleotide methylation (e.g., determining percent methylation, presence and amount of DNA carrying a mutation, etc.) typically requires normalization to a control nucleic acid, e.g., an endogenous invariant gene having known features (e.g., known sequence, known copy-number per cell). Normalizing controls for sample-to-sample variations that may occur in, for example, sample processing, assay efficiency, etc., allows accurate sample-to-sample data comparison. Further, materials and methods to validate or confirm completion of conversion assays in the course of methylation studies offers confidence in the output of the assays.

### SUMMARY

In one aspect, described herein is a composition comprising four or more unique oligonucleotides, each of the four or more oligonucleotides comprising a unique nucleotide sequence and a unique number of methylated bases, wherein the unique number of methylated bases is about 0% to 100% of the unique nucleotide sequence of each of the four or more oligonucleotides.

In another aspect, described herein is a composition comprising four or more oligonucleotides each having a unique pattern of methylated bases in methylation motifs, wherein each of the unique oligonucleotides have a percent of methylated bases between 0.1% and 100%.

The disclosure also provides a method, comprising: a) adding a known amount of the composition described herein having a predetermined percent methylation to a test sample comprising a target nucleic acid; b) determining the percent methylation of the target nucleic acid; and c) determining the percent methylation of oligonucleotides in the composition.

### BRIEF DESCRIPTION OF THE DRAWING

FIG. 1 shows a histogram of control aligned methylated fragments by methylation percentage where methylation score bins are represented as dashed lines.

FIG. 2 shows a histogram of coverage count depth by base position across an aligned control sequence.

FIG. 3 shows a histogram of coverage counts depth for cysteine base residues by base position across an aligned control sequence.

FIG. 4 shows a histogram of coverage count depth for cysteine base residues by base position across an aligned control sequence.

FIG. 5 shows a quality score calling cutoff plotted against the respective mean square contingency coefficient (MCC) score for all methylated molecules relative to their respective correct control call.

FIG. 6 shows a quality score calling cutoff plotted against the respective mean F1 score for all methylated molecules relative to their respective correct control call

### DETAILED DESCRIPTION

The present application is based, at least in part, on the discovery that incorporation of methylated control oligonucleotides that are methylated along a gradient (e.g., methylation of varying percentages (including and) between 0% and 100%) in a sample allows for highly-sensitive detection of methylated oligonucleotides in a sample. Potential variation in methylation conversion efficiency among samples can lead to inaccurate or inconsistent read outs. Further, failure to understand the limits of detection of a particular assay can cast doubt on reliability of the read out.

### Composition

The disclosure provides a composition comprising multiple oligonucleotides each having a unique nucleotide sequence and a unique number of methylated bases comprising about 0% to 100% of the unique nucleotide sequence. The composition, thus, may serve as a gradient-based control composition for methylation assays to improve confidence in determining methylation patterns and methylation content of oligonucleotides from patient samples. In this respect, the disclosure also provides methods of using the composition described herein. To illustrate, the composition described herein can be used to (1) demonstrate successful amplification of highly methylated fragments or low methylated fragments; (2) offer insight into the limit of detection of assays (e.g., the methods described herein established that methylation patterns in oligonucleotides could be identified at a resolution of as low as 5% using Oxford Nanopore Technology (ONT) for highly methylated fragments and for low methylated fragments); (3) choose an ideal quality cutoff value for methylation calling; (4) determine the motif accuracy of methylation calling; and (5) act as a marker and comparator for estimating error rate, fragment size, run molarity, and conversion efficiency.

In one aspect, described herein is a composition comprising four or more oligonucleotides, each of the four or more oligonucleotides comprising a unique nucleotide sequence and a unique percentage of methylated bases, wherein the unique percentage of methylated bases ranges from about 0% to 100% of the unique nucleotide sequence of each of the four or more oligonucleotides. The oligonucleotides in the composition can be single-stranded or double stranded.

The term “methylation” or methylated” refers to a process by which a methyl group is added to an oligonucleotide (e.g., DNA molecule). For example, a hydrogen atom on the pyrimidine ring of a cytosine base can be converted to a methyl group, forming 5-methylcytosine. Methylation and hydroxymethylation tend to occur at dinucleotides of cytosine and guanine referred to herein as “CpG sites”. The term “CpG site” refers to a region of a oligonucleotide where a cytosine nucleotide is followed by a guanine nucleotide in the linear sequence of bases along its 5′ to 3′ direction. “CpG” is a shorthand for 5′-C-phosphate-G-3′ that is cytosine and guanine separated by only one phosphate group. Cytosines in CpG dinucleotides can be methylated to form 5-methylcytosine. Most cytosines within the CpG dinucleotides are methylated in the human genome, however some remain unmethylated in specific CpG dinucleotide rich genomic regions, known as CpG islands (see, e.g., Antequera et al. (1990) Cell 62:503-514).

In some instances, a CpG island can be at least 500 base pairs in length, where the G:C content of the region is at least 55%, and the ratio of observed CpG frequency over expected frequency is 0.65. The observed CpG frequency over expected frequency can be calculated according to any suitable method, including the method provided in Gardiner-Garden et al (1987) J. Mol. Biol. 196:261-281. For example, the observed CpG frequency over expected frequency can be calculated according to the formula R=(A×B)/(C×D), where R is the ratio of observed CpG frequency over expected frequency, A is the number of CpG dinucleotides in an analyzed sequence, B is the total number of nucleotides in the analyzed sequence, C is the total number of C nucleotides in the analyzed sequence, and D is the total number of G nucleotides in the analyzed sequence. Methylation state is typically determined in CpG islands, e.g., at promoter regions. It will be appreciated though that other sequences in the human genome are prone to DNA methylation such as CpA and CpT (see Ramsahoye (2000) Proc. Natl. Acad. Sci. USA 97:5237-5242; Salmon and Kaye (1970) Biochim. Biophys. Acta. 204:340-351; Grafstrom (1985) Nucleic Acids Res. 13:2827-2842; Nyce (1986) Nucleic Acids Res. 14:4353-4367; Woodcock (1987) Biochem. Biophys. Res. Commun. 145:888-894).

In some embodiments, the methylated base is an adenine or a cytosine. In some embodiments, the adenine methylation comprises N6-methyladenine (6 mA). In some embodiments, the cytosine methylation comprises 5-methylcytosine (5mC) or N4-methylcytosine (4mC). In some embodiments, the cytosine methylation comprises Dcm methylation, DNMT1 methylation, DNMT3A methylation or DNMT3B methylation. In some embodiments, the cytosine methylation comprises CpG methylation, CpA methylation, CpT methylation, CpC methylation or a combination thereof. In some embodiments, the cytosine methylation comprises CpG methylation.

In another aspect, described herein is a composition comprising four or more oligonucleotides, each having a unique nucleotide sequence and a unique percentage of methylation motifs within the nucleotide sequence, wherein the unique percentage of methylation motifs of the nucleotide sequence ranges from 0% and 100% of the unique nucleotide sequence (for example, the percentage of nucleotide bases in the sequence that comprise any of the methylation motif(s)). The term “methylation motif” refers to a contiguous sequence of nucleotides that is methylated. In some embodiments, multiple methylation motifs of a unique nucleotide sequence are methylated. In some embodiments, the methylation motifs are at least 10 base pairs away from a 5′ end of the oligonucleotide. In some embodiments, the methylation motifs are at least 10, 15, 20, 25, 30, 35, 40, 45, or 50 base pairs away from the 5′ end of the oligonucleotide. In some embodiments, the methylation motifs are at least 10 base pairs away from a 3′ end of the oligonucleotide. In some embodiments, the methylation motifs are at least 10, 15, 20, 25, 30, 35, 40, 45, or 50 base pairs away from the 3′ end of the oligonucleotide. Exemplary methylation motifs include, but are not limited to, the motifs identified in Table 1.

TABLE 1
Exemplary Methylation Motifs

| SEQ ID NO | Sequence |
|---|---|
| 1 | ATGGAGTCTCGC |
| 2 | ACAGAGAATTAC |
| A | CCCCGCCC |
| B | CCCGCC |
| 3 | CGCAGCGCGG |
| C | TGGGCAACA |
| D | CGCCGCGCA |
| E | CTCTGCGCA |
| F | CGCGCGCG |
| 4 | GCGCGCGCGC |
| G | GCCTCAGCC |
| H | CTCACC |
| 5 | CNNNNGNNGG |
| 6 | CACNNGGGCG |

In some embodiments, the unique percentage of methylated bases within a nucleotide sequence is about 0% to about 100%, or about 0.001% to 90%, or about 0.1% to 90%, or about 0.5% to 85%, or about 1% to 30%, or about 1% to 90%, or about 10% to about 90%, or about 20% to about 100%, or about 50% to about 100% of the unique nucleotide sequence of an oligonucleotide in the composition. In some embodiments, the unique percentage of methylated bases within a nucleotide sequence is about 0%, about 0.001%, about 0.01%, about 0.1%, about 0.5%, about 1%, about 1.5%, about 2%, about 2.5%, about 3%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% of the unique nucleotide sequence in the composition (or any range wherein two recited percentages are endpoints of the range).

In some embodiments, the composition comprises a first oligonucleotide comprising a first nucleotide sequence, wherein 0.1% of the bases of the first nucleotide sequence are methylated; a second oligonucleotide comprising a second nucleotide sequence, wherein 0.5% of the bases of the second nucleic acid sequence are methylated; a third oligonucleotide comprising a third nucleotide sequence, wherein 15% of the bases of the third nucleotide sequence are methylated; and a fourth oligonucleotide comprising a fourth nucleotide sequence, wherein 40% of the bases of the fourth nucleotide sequence are methylated. In some embodiments, the composition further comprises a fifth oligonucleotide comprising a fifth nucleotide acid, wherein 80% of the bases of the fifth nucleotide sequence are methylated.

In some embodiments, the composition comprises a first oligonucleotide comprising a first nucleotide sequence, wherein 5% of the bases of the first nucleotide sequence are methylated; a second oligonucleotide comprising a second nucleotide sequence, wherein 10% of the bases of the second nucleotide sequence are methylated; a third oligonucleotide comprising a third nucleotide sequence, wherein 55% of the bases of the third nucleotide sequence are methylated; and a fourth oligonucleotide comprising a fourth nucleotide sequence, wherein 85% of the bases of the fourth nucleotide sequence are methylated.

In some embodiments, the unique percentage of methylation motifs within a unique nucleotide sequence is about 0% to about 100%, or about 1% to 30%, or about 1% to 90%, or about 10% to about 90%, or about 20% to about 100%, or about 50% to about 100% of the unique nucleotide sequence in the composition. In some embodiments, the unique percentage of methylation motifs within the unique nucleotide sequence is about 0%, about 0.1%, about 0.5%, about 1%, about 1.5%, about 2%, about 2.5%, about 3%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% of the unique nucleotide sequence in the composition (or any range wherein two recited percentages are endpoints of the range).

The compositions described herein can comprise any number of oligonucleotides having a unique nucleotide sequence and a unique percentage of methylated bases (or unique percentage of methylated bases within a methylation motif). For example, in some embodiments, the composition comprises at least 4, at least 5, at least 6, at least 7, at least 8, at least 9, at least 10, at least 15, at least 20, at least 25, at least 30, at least 40, at least 50, at least 60, at least 70, at least 80, at least 90, at least 100, or more unique oligonucleotides, each having a unique nucleotide sequence and a unique percentage of methylated bases of the unique nucleotide sequence in the composition (or unique percentage of methylated bases within a methylation motif of the unique nucleotide sequence in the composition).

Optionally, the oligonucleotides in the composition have a methylation profile along a linear, logarithmic, exponential, semi-log scale, and/or sigmoidal curve. To illustrate, control oligonucleotides are used having varying amounts of methylation and/or varying degrees of methylation. For instance, control oligos may comprise the sequence ATGCCGT, ATGC*CGT, or ATGC*C*GT, wherein the C* represents a methylated C. In this example, ATGCCGT would be 0% methylated, ATGC*CGT would be 50% methylated, ATGCC*GT would be 50% methylated but at a different position, and ATGC*C*GT would be 100% methylated. The degree of methylation, the positions of methylation, and the amounts of oligonucleotides are adjusted as needed to generate a linear curve, a logarithmic curve, an exponential curve, and the like.

The compositions described herein comprise oligonucleotides that are not naturally-occurring. For example, in some embodiments, the oligonucleotides share less than 25% sequence identity along a contiguous region of a sequence present in a human gene. For example, in some embodiments, each of the oligonucleotides in a composition share less than 25%, less than 24%, less than 23%, less than 22%, less than 21%, less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, or less than 1% sequence identity along a contiguous region of a sequence within a human gene.

The oligonucleotides described herein could be of any length. For example, in some embodiments, the oligonucleotides are about 50 nucleotides to about 4000 nucleotides in length. In some embodiments, the oligonucleotides are about 50 nucleotides to about 300, or about 100 to about 400, or about 200 to about 600 nucleotides in length. For example, in some embodiments, the oligonucleotides in a composition are 50, 100, 150, 200, 250, 300, 350, 400, 450, 500, 600, 700, 800, 900, 1000, 1500, 2000, 2500, 3000, 3500 or 4000 nucleotides in length. In some embodiments, the composition comprises at least four oligonucleotides (each having a unique nucleotide sequence and a unique percentage of methylated bases) of varying lengths. For example, in some embodiments, the composition comprises a first oligonucleotide that is 100 nucleotides in length; a second oligonucleotide that is 120 nucleotides in length; a third oligonucleotide that is 150 nucleotides in length; a fourth oligonucleotide that is at least 180 nucleotides in length; and a fifth oligonucleotide that is at least 200 nucleotides in length.

In some embodiments, each unique oligonucleotide in the composition is present in a different concentration. Merely to illustrate, an oligonucleotide comprising a sequence which is 5% methylated may be present in the composition at a first concentration, and an oligonucleotide sequence which is 10% methylated may be present in the composition at a second concentration, and so on.

The oligonucleotides described herein may optionally include features such as a hairpin and/or a barcode. “Hairpin”, “hairpin loops,” and “stem-loops” are nucleic acid secondary structural elements which form via intramolecular base pairing in single-stranded DNA or RNA. A hairpin occurs when two regions of the same nucleic acid strand, usually complementary in nucleotide sequence, base-pair to form a double helix that ends in a loop of unpaired nucleotides. The term “barcode” refers to a unique nucleic acid that can be used to identify the unique nucleic acid sequence. In some embodiments, a barcode sequence is adjacent (e.g., immediately next to) or near a hairpin structure.

The composition comprising the methylation control oligonucleotides can comprise any acceptable carrier, diluent or excipient known in the art. Exemplary buffers used in formulation of the disclosure include acetate, sulfate, hydrochloride, phosphate or free acid forms of Tris-(hydroxymethyl)aminomethane (TRIS), although alternative buffers of the same approximate ionic strength and pKa as TRIS may be used with equivalent results. In addition to the buffer salts, cofactor salts such as those of potassium (e.g., potassium chloride or potassium acetate) and magnesium (e.g., magnesium chloride or magnesium acetate) are optionally included in the compositions.

### Methods

Provided herein is method, comprising a) adding a known amount of the composition described herein (i.e., a control composition) having a predetermined percent methylation to a test sample comprising a target nucleic acid; b) determining the percent methylation of the target nucleic acid; and c) determining the percent methylation of oligonucleotides in the control composition. Also described herein is a method comprising a) adding a known amount of a composition comprising four or more oligonucleotides to a test sample, each of the four or more oligonucleotides comprising a unique nucleotide sequence and a unique percentage of methylated bases, wherein the unique percentage of methylated bases ranges from about 0% to 100% of the unique nucleotide sequence of each of the four or more oligonucleotides; (b) identifying the percent methylation of each of the oligonucleotides in the composition; and (c) determining a methylated cutoff value based on step (b). The composition of the method is described above, and any disclosure relating to the composition above applies to the control composition of the methods described herein.

The term “test sample” refers to a sample comprising a target nucleic acid. The term “target nucleic acid” refers to a nucleotide sequence for which it is desirable to determine a methylation status. In various aspects, the test sample is obtained from a human subject, such as a human subject suffering from or at risk of suffering from a disease or disorder (such as cancer). In some embodiments, the method further comprises identifying the percent methylation of the target nucleic acid in the sample. Exemplary test samples include, but are not limited to blood, saliva, liquid biopsy, and mucus.

Methylation of a nucleotide sequence can be determined by any suitable means. For example, in some embodiments, methylation is determined using short-read sequencing or digital PCR. In some embodiments, methylation is determined by Illumina dye sequencing or Sanger sequencing. For example, next generation sequencing (NGS) techniques such as sequencing-by-synthesis technology (Illumina), pyrosequencing (454 Life Sciences), ion semiconductor technology (Ion Torrent sequencing), single-molecule real-time sequencing (Pacific Biosciences), sequencing by ligation (SOLID sequencing), or nanopore sequencing (Oxford Nanopore Technologies) is performed. In some embodiments sequencing is performed on a next generation sequencer (e.g., an Illumina HiSeq 4000, Illumina NovaSeq 6000, Oxford Nanopore, Biomodal) to a unique on-target depth selected by the user. In some embodiments, massively parallel sequencing is performed using sequencing-by-synthesis with reversible dye terminators.

Analysis of site-specific methylation patterns can be performed using, e.g., bisulfite conversion methods and non-bisulfite based methods. The bisulfite conversion method relies on treatment of DNA samples with sodium bisulfite which converts unmethylated cytosine to uracil, while methylated cytosines are maintained. This conversion results in a change in the sequence of the original DNA. Analysis of the sequence of the resulting DNA allows the determination of which cytosines in the DNA were methylated. There are several methodologies utilized for the analysis of bisulfite converted DNA including sequencing, methylation-specific PCR, COBRA (Combined Bisulfite Restriction Analysis), methylation-sensitive single nucleotide primer extension, and methylation-sensitive single-strand conformation analysis.

The most frequently used method for analyzing a nucleic acid for the presence of 5-methylcytosine is based upon the bisulfite method described by Frommer et al. for the detection of 5-methylcytosines in DNA (Frommer et al. (1992) Proc. Natl. Acad. Sci. USA 89:1827-31 explicitly incorporated herein by reference in its entirety for all purposes) or variations thereof. The bisulfite method of mapping 5-methylcytosines is based on the observation that cytosine, but not 5-methylcytosine, reacts with hydrogen sulfite ion (also known as bisulfite). The reaction is usually performed according to the following steps: first, cytosine reacts with hydrogen sulfite to form a sulfonated cytosine. Next, spontaneous deamination of the sulfonated reaction intermediate results in a sulfonated uracil. Finally, the sulfonated uracil is desulfonated under alkaline conditions to form uracil. Detection is possible because uracil base pairs with adenine (thus behaving like thymine), whereas 5-methylcytosine base pairs with guanine (thus behaving like cytosine). This makes the discrimination of methylated cytosines from non-methylated cytosines possible by, e.g., bisulfite genomic sequencing (Grigg G, & Clark S, Bioessays (1994) 16:431-36; Grigg G, DNA Seq. (1996) 6:189-98), methylation-specific PCR (MSP) as is disclosed, e.g., in U.S. Pat. No. 5,786,146, or using an assay comprising sequence-specific probe cleavage, e.g., a QuARTS flap endonuclease assay (see, e.g., Zou et al. (2010) “Sensitive quantification of methylated markers with a novel methylation specific technology” Clin Chem 56: A199; U.S. Pat. No. 8,361,720, and U.S. Patent Publication Nos. US 2012/0122088; US 2012/0122106 and US 2014/0087382, incorporated by reference herein).

The bisulfite technique typically involves amplifying short, specific fragments of a known nucleic acid subsequent to a bisulfite treatment, then assaying the product by sequencing (Olek & Walter (1997) Nat. Genet. 17:275-6) or a primer extension reaction (Gonzalgo & Jones (1997) Nucleic Acids Res. 25:2529-31; WO 95/00669; U.S. Pat. No. 6,251,594) to analyze individual cytosine positions. Some methods use enzymatic digestion (Xiong & Laird (1997) Nucleic Acids Res. 25:2532-4). Detection by hybridization has also been described in the art (Olek et al., WO 99/28498). Additionally, use of the bisulfite technique for methylation detection with respect to individual genes has been described (Grigg & Clark (1994) Bioessays 16:431-6; Zeschnigk et al. (1997) Hum Mol Genet. 6:387-95; Feil et al. (1994) Nucleic Acids Res. 22:695; Martin et al. (1995) Gene 157:261-4; WO 9746705; WO 9515373).

The term “bisulfite reagent” refers to a reagent comprising bisulfite, disulfite, hydrogen sulfite, or combinations thereof, useful as disclosed herein to distinguish between methylated and unmethylated CpG dinucleotide sequences. Methods of said treatment are known in the art (e.g., PCT/EP2004/011715 and WO 2013/116375, each of which is incorporated by reference in its entirety). In some embodiments, bisulfite treatment is conducted in the presence of denaturing solvents such as but not limited to n-alkylenglycol or diethylene glycol dimethyl ether (DME), or in the presence of dioxane or dioxane derivatives. In some embodiments the denaturing solvents are used in concentrations between 1% and 35% (v/v). In some embodiments, the bisulfite reaction is carried out in the presence of scavengers such as but not limited to chromane derivatives, e.g., 6-hydroxy-2,5,7,8,-tetramethylchromane 2-carboxylic acid or trihydroxybenzone acid and derivates thereof, e.g., Gallic acid (see: PCT/EP2004/011715, which is incorporated by reference in its entirety). In certain preferred embodiments, the bisulfite reaction comprises treatment with ammonium hydrogen sulfite, e.g., as described in Hayatsu, H., Proc. Jpn. Acad., Ser. B 84, No. 8:321 (2008).

Various methylation assay procedures can be used in conjunction with bisulfite treatment. These assays allow for determination of the methylation state of one or a plurality of CpG dinucleotides (e.g., CpG islands) within a nucleic acid sequence. Such assays involve, among other techniques, sequencing of bisulfite-treated nucleic acid, PCR (for sequence-specific amplification), Southern blot analysis, and use of methylation-sensitive restriction enzymes.

Methylation-Specific PCR (MS-PCR) is the most commonly used technique for analysis of methylation. MS-PCR is utilized to determine the methylation status of specific cytosines following conversion of unmethylated cytosines to uracil by bisulfite conversion (Herman et al., PNAS 1996 Sep. 3; 93(18): 9821-6). The methylation status of specific cytosines can be determined by utilizing primers that are specific for the cytosine of interest. The differences in sequences following conversion allow different primer sets to determine whether the initial sequence was methylated. Melting curve Methylation Specific PCR (McMS-PCR) replaced sequence analysis of the resulting PCR products, with the more efficient process of melt curve analysis (Smith et al., BMC Cancer. 2009 Apr. 24; 9:123). Differences in the melting temperature of the products are due to the sequence differences resulting from bisulfite conversion of methylated versus unmethylated DNA samples. Another method for analyzing MS-PCR products using melting characteristics involves the use of denaturing high-performance liquid chromatography (Baumer, Methods. 2002 June; 27 (2): 139-43). In this method, MS-PCR is carried out under conditions that will amplify both alleles (converted and unconverted cytosines). The products of MS-PCR are analyzed by HPLC under denaturing conditions, allowing the resolution of different products based on sequence differences due to bisulfite conversion.

One version of MS-PCR, called MethyLight (Eads et al., Nucleic Acids Res. 2000 Apr. 15; 28(8): E32), involves the use of fluorescence-based real-time quantitative PCR to allow both detection and quantitation of the converted products in one step. The major drawback of these techniques is the necessity to design primers specific for each methylation site that are based on the different converted sequence possibilities. An additional modification to the MethyLight protocol involves using an additional fluorescent probe directed against unconverted DNA. This protocol, ConLight-MSP, was developed to address the issue of overestimation of methylation due to incomplete conversion of DNA by bisulfite (Rand et al., Methods. 2002; 27:114-20). A second method aimed at addressing the problem of incomplete bisulfite conversion is bisulfite conversion-specific Methylation-Specific PCR (BS-MSP) (Sasaki et al., Biochem Biophys Res Commun. 2003 Sep. 19; 309(2): 305-9). In this technique, two rounds of PCR are carried out following bisulfite conversion of DNA. In the first round, primers are utilized that do not contain CpG's, but do contain cytosines at the 3′ position. Thus, only fully converted DNA will be amplified in the first round of amplification. A second, traditional MSP amplification is subsequently carried out to amplify the CpG's of interest. This will result in a lower level of background amplification of sites with incomplete conversion of DNA, and a more accurate determination of the level of methylation in the sample.

Other methods for site-specific methylation analysis include COBRA, Methylation-sensitive single nucleotide primer extension (MS-SNuPE), and methylation-sensitive single-strand conformation analysis (MS-SSCA). COBRA combines the techniques of bisulfite conversion with methylation-sensitive restriction endonuclease analysis (described below) to enable highly specific, highly sensitive quantitation of methylation sites contained within recognition sites for methylation-sensitive restriction enzymes (Xiong and Laird, Nucleic Acids Res. 1997 Jun. 15; 25(12): 2532-4). Melting curve combined bisulfite restriction analysis (McCOBRA) was developed to allow analysis of bisulfite converted DNA without gel electrophoresis (Akey et al., 2002, Genomics, 80:376-384). In this procedure, bisulfite converted DNA is amplified by PCR with specific primer pairs surrounding a potential methylation site. The resulting PCR products are digested with a restriction site that will only recognize and cut DNA that was originally methylated. Melt curve analysis will yield two peaks, based on the size difference of the cut versus uncut DNA, and allow the determination of the methylation status of that site in the original DNA. Another variation of COBRA, termed Pyro-sequencing methylation analysis (PyroMethA) involves the use of the Pyrosequencing reaction to determine methylation status in place of the restriction analysis used in COBRA (Tost et al., BioTechniques 40 6 (2006): 721-2, 724, 726). MS-SNUPE combines MS-PCR amplification of bisulfite converted DNA with single nucleotide extension of MS-PCR products to incorporate radio-labeled C (methylated) or T (unmethylated) that can be detected using a phosphoimager (Gonzalgo and Jones, Nucleic Acids Res., 25(12): 2529-2531, 1997). The ratio of C/T incorporation will indicate the level of methylation at a particular site. Finally, MS-SSCA utilizes bisulfite converted DNA with single-stranded conformational polymorphism (SSCP) analysis to detect sequence differences through changes in the migration of the molecules during electrophoresis (Burri and Chaubert, Biotechniques. 1999; 26:232-234).

In some embodiments, percent methylation of a nucleotide sequence in a test sample is determined by 5hmC sequencing, CpG sites sequencing (MCTA-seq), methylated DNA immunoprecipitation (cfMeDIP-seq), methylation ddPCR (qMSP), methylation detection using ionic current signal from Nanopore sequencing, targeted bisulfite sequencing, TET-assisted pyridine borane sequencing (TAPS), whole genome bisulfite sequencing (WGBS), or any combination thereof.

In some embodiments, the methylation control oligonucleotides contained in the composition described herein are processed in parallel with test samples, for example clinical or experimental samples. Methylation controls processed in parallel with test samples can be used to generate a calibration curve that is used to determine the methylation status of a target nucleic acid sequence in a test sample. For example, the test sample can be a clinical sample comprising oligonucleotides obtained from cells from a human host (e.g., tumor cells or a liquid tumor sample). In alternative embodiments, the composition described herein is added directly to a test sample, such as a clinical or experimental test sample, and are used as an internal standard of methylation status.

In some embodiments, the composition comprising the methylation control oligonucleotides described herein is processed in parallel with, but separate from (i.e., not “spiked” into), a test sample, optionally to generate a high throughput sequencing library and a collection of reads therefrom. In some embodiments, normalization controls are added to a methylation control sample and a test sample, and are used to normalize the reads from the methylated control sample and the test sample.

In some embodiments, the methods described herein comprise sequencing nucleotide sequences (e.g., target nucleotide sequences and methylated control nucleotide sequences) in the test sample by next generation sequencing technology (NGS) in which clonally amplified DNA templates or single DNA molecules are sequenced in a massively parallel fashion within a flow cell (e.g., as described in Volkerding et al. Clin Chem 55:641-658 [2009]; Metzker M Nature Rev 11:31-46 [2010]). In addition to high-throughput sequence information, NGS provides digital quantitative information, in that each sequence read is a countable “sequence tag” representing an individual clonal DNA template or a single DNA molecule.

In some embodiments, the methods described herein comprise sequencing nucleotide sequences (e.g., target nucleotide sequences and methylated control nucleotide sequences) in the test sample by 454 sequencing (Roche) (e.g. as described in Margulies, M. et al. Nature 437:376-380 [2005]). In 454 sequencing DNA fragments of approximately 300-800 base pairs, are blunt-ended and adaptors are ligated to the ends of the fragments. The adaptors serve as primers for amplification and sequencing of the fragments. The fragments can be attached to affinity moieties including but not limited to DNA capture beads, e.g., streptavidin-coated beads using, e.g., Adaptor B, which contains 5′-biotin tag. The fragments attached to the beads are PCR amplified within droplets of an oil-water emulsion. The result is multiple copies of clonally amplified DNA fragments on each bead. In the second step, the beads are captured in wells (pico-liter sized). Pyrosequencing is performed on each DNA fragment in parallel. Addition of one or more nucleotides generates a light signal that is recorded by a CCD camera in a sequencing instrument. The signal strength is proportional to the number of nucleotides incorporated.

Pyrosequencing makes use of pyrophosphate (PPi) which is released upon nucleotide addition. PPi is converted to ATP by ATP sulfurylase in the presence of adenosine 5′ phosphosulfate. Luciferase uses ATP to convert luciferin to oxyluciferin, and this reaction generates light that is discerned and analyzed. 454 Sequencing can produce reads that are between 100-1000 bp in length.

### Cancer

Aberrant DNA methylation is associated with many cancers including breast, liver, bone, and colon, and has been identified as a biomarker for early cancer screening. Liquid biopsies are rich samples for screening for DNA methylation in cancer-derived DNA. Epigenetic modifications are used to assign a tissue of origin to the cancer to direct confirmatory diagnostic procedures. However, accurate assessment of methylation is not trivial, and methods like bisulfite sequencing can result in DNA damage in the sample.

In some embodiments, a test sample is derived from blood, serum, plasma, gastric secretions, pancreatic juice, biopsy samples, e.g., from gastrointestinal, lung, and other cancers, microdissected cells from a biopsy, cells sloughed into the lumen, and/or cells recovered from stool. In some embodiments, the subject is human. The sample may include cells, secretions, or tissues from, for example, the lung, liver, bile ducts, pancreas, stomach, colon, rectum, esophagus, small intestine, appendix, duodenum, polyps, gall bladder, anus, and/or peritoneum. In some embodiments, the sample comprises cellular fluid, ascites, urine, feces, pancreatic fluid, fluid obtained during endoscopy, blood, mucus, or saliva. In some embodiments, the sample is a stool sample.

Patient samples can be obtained by any number of means, including those known in the art. For instance, urine and fecal samples are easily attainable, while blood, ascites, serum, or pancreatic fluid samples can be obtained parenterally by using a needle and syringe, for instance. Cell free or substantially cell free samples can be obtained by subjecting the sample to various techniques known to those of skill in the art which include, but are not limited to, centrifugation and filtration. Although it is generally preferred that invasive techniques are avoided in obtaining the sample, it still may be preferable to obtain samples such as tissue homogenates, tissue sections, and biopsy specimens. The technology is not limited in the methods used to prepare the samples and provide a nucleic acid for testing. For example, in some embodiments, oligonucleotides (e.g., DNA) are isolated from a stool sample or from blood or from a plasma sample using direct gene capture, e.g., as detailed in U.S. Pat. Nos. 8,808,990 and 9,169,511, and in International Patent Publication No. WO 2012/155072, or by a related method.

In various aspects, the test sample contains a target nucleic acid derived from a patient that is suffering from cancer or who is at risk of suffering from cancer. The assays described herein may be employed in protocol to determine or characterize a subject's diagnostic status, a type of cancer, a location of cancer, a primary origin of a cancer, a cancer stage, a cancer prognosis, and/or one or more additional characteristics of a cancer (e.g., tumor characteristics such as morphology, heterogeneity, size, etc.). As used herein, the terms “patient” and “subject” are used interchangeably and may be taken to mean any living or non-living organism including, but not limited to, a human (e.g., a male human, female human, fetus, pregnant female, child, or the like), a non-human mammal, or a non-human animal. Any human or non-human animal can serve as a subject, including but not limited to mammal, reptile, avian, amphibian, fish, ungulate, ruminant, bovine (e.g., cattle), equine (e.g., horse), caprine and ovine (e.g., sheep, goat), swine (e.g., pig), camelid (e.g., camel, llama, alpaca), monkey, ape (e.g., gorilla, chimpanzee), ursid (e.g., bear), poultry, dog, cat, mouse, rat, fish, dolphin, whale and shark. In some embodiments, a subject is a male or female of any age (e.g., a man, a woman, or a child).

In some embodiments, the cancer is lung cancer (e.g., non-small-cell lung cancer). In some embodiments, the cancer is breast cancer. Additional non-limiting examples of cancers include ovarian cancer, cervical cancer, uveal melanoma, colorectal cancer, chromophobe renal cell carcinoma, liver cancer, endocrine tumor, oropharyngeal cancer, retinoblastoma, biliary cancer, adrenal cancer, neural cancer, neuroblastoma, basal cell carcinoma, brain cancer, non-clear cell renal cell carcinoma, glioblastoma, glioma, kidney cancer, gastrointestinal stromal tumor, medulloblastoma, bladder cancer, gastric cancer, bone cancer, thymoma, prostate cancer, clear cell renal cell carcinoma, skin cancer, thyroid cancer, sarcoma, testicular cancer, head and neck cancer (e.g., head and neck squamous cell carcinoma), meningioma, peritoneal cancer, endometrial cancer, pancreatic cancer, mesothelioma, esophageal cancer, small cell lung cancer, HER2 negative breast cancer, ovarian serous carcinoma, HR+ breast cancer, uterine serous carcinoma, uterine corpus endometrial carcinoma, gastroesophageal junction adenocarcinoma, gallbladder cancer, chordoma, and papillary renal cell carcinoma.

In some embodiments, the cancer is a particular stage of a particular type of cancer. In some such embodiments, the stage of the particular type of cancer is a stage of cancer the subject was diagnosed with prior to conducting a method described herein. Cancer is typically staged to determine the extent of its spread and to guide treatment decisions. The stage of cancer refers to the extent to which it has grown and spread from its original location. Each cancer has its own criteria for determine stage but generally relies on a determination of the size of the primary tumor (T) and whether it has invaded nearby tissues, evaluation of lymph node involvement (N) to find indications of whether cancer has spread to nearby lymph nodes, and assessment of distant metastasis (M), which indicates whether the cancer has spread to distant organs or tissues. Metastasis means that cancer has spread from the primary site to other parts of the body. In some embodiments the staging system used is the cancer TNM system, which combines the T, N, and M information to assign a stage. In some embodiments the stages are denoted using Roman numerals (I, II, III, IV) and may have subcategories (e.g., stage IIA, stage IIB) to provide more precise information. In a brief overview of these stages, in stage 0, the cancer is in situ, meaning it is confined to the layer of cells where it began and has not invaded nearby tissues, in stage I: the cancer is localized and small in size, in stage II, the cancer may be larger and/or have spread to nearby lymph nodes, but it is still relatively localized, in stage III, the cancer has typically spread further into nearby tissues and may involve more lymph nodes, in stage IV, the cancer has spread to distant organs or tissues, indicating metastasis. This is often the most advanced stage. The specific criteria for each stage can vary depending on the type of cancer. See, for example, details of TNM staging for breast cancer in Part et al., 2011, “Clinical relevance of TNM staging system according to breast cancer subtypes,” Annals of Oncology 22(7), pp. 1554-1560, which is hereby incorporated by reference. Additionally, some cancers have their own staging systems tailored to their characteristics. See the world wide web at cancer.gov/about-cancer/diagnosis-staging/staging. The patient may be at risk for molecular residual disease (MRD), also called minimal residual disease or minimal residual disease, and the sequencing may be performed with the goal of detecting whether the patient has MRD. See also, U.S. Patent Publication No. 2025/0061972.

Circulating tumor DNA (ctDNA) is a promising biomarker that carries cancer-specific genetic and epigenetic aberrations, which therefore can be used as a surrogate source of tumor DNA in cancer diagnosis and prognosis prediction. For many cancer types, symptoms are often unapparent at an early stage; therefore, many efforts have been made to develop reliable, noninvasive, and cost-effective early detection methods. Currently, the potential of ctDNA biomarkers is under intensive investigation for detecting early-stage cancer, such as somatic mutations, single-nucleotide variants, and somatic copy-number aberrations.

### EXAMPLE

### Example 1—Amplification of Variously Methylated DNA Fragments

### Background

A goal of the experiments described herein was to demonstrate successful amplification of highly methylated DNA fragments and to showcase a nanopore method of methylation calling. To achieve this result, a variety of techniques was used. A new technique for controlling methylation calling also was employed, namely utilizing oligos with known methylated bases added to samples prior to sequencing as a sequencing control. The oligonucleotides at known initial concentrations were then used to:
1. Demonstrate successful amplification of highly methylated fragments. The initial starting concentrations of all oligonucleotides prior to the use of methylation specific antibodies for isolation was 1:1:1:1:1 for all 5 oligo sequences used.
2. Show that methylation patterns in fragments could be shown down to a resolution of 5% both with nanopore technology (ONT) for highly methylated fragments and for low methylated fragments. Each fragment had a unique pattern of known methylated cytosine.
3. Choose an ideal quality cutoff value for methylation calling on the experimental parent strands. Since their methylation status remained unknown, and because an amplification step was used, the resulting DNA quality for calling a methylated base should be selected that maximizes both precision and accuracy.
4. Determine the motif accuracy of the method of methylation calling utilized. The base caller used for ONT relies on a model to correctly call base quality. Therefore, the base caller may miss methylation calls after certain base sequences. It is desirable to detect these low quality calls.
5. Similar to a PhiX internal sequencing control on an Illumina run (or sequencing on another platform), the oligonucleotide controls in the composition described herein also can act as a marker and yard stick for estimating error rate, fragment size, run molarity, and conversion efficiency.

### Methods

The analysis pipeline was built on the .sh scripts and utilized Darado for base calling and alignment. The next steps taken were to isolate reads aligning to the barcode oligos.

The primary oligonucleotide used for alignment was as follows:

(SEQ ID NO: 7)
TCGATCGTTCGACCGACTCGGCGATAATGTTCGCGACGCTCGCTCTGAAA
TTCGCGAACGCCACGCCTCGAGTGAAGACGCGATTGCGCACCATCGCGCT
GCGCCGTCACGCGCGTAGAAACTCGTCGCG.

Each of the methylation fragments had (1) a consistent nucleic acid sequence, except for the location of the methylated bases within the sequence and (2) a unique barcode. Since these were made as oligonucleotides and were then hybridized with their unmethylated complement, only primary forward alignment fragments were utilized, not fragments that align to the reverse complement of the fragment, as the reverse complement was unmethylated. This was achieved by limiting to read with a .sam flag of 0. Next, all barcode sam files on the run were concatenated to achieve sufficient representation of samples, since there were few fragments in the final results.

The Sequence Alignment/Map (SAM) format stores methylation and other calls in their own section with the first section indicating the position of the methylated call and the second section indicating the quality score of that position. This is a number from 0-255, with 255 being the highest score possible.

To fingerprint these methylation calls, all aligned residues were identified and their harmonic mean of the precision and recall (F1) and mean square contingency coefficient (MCC) values were calculated and compared against all possible methylation references 5%-90%. To calculate these metrics, we determined that each cytosine residue's positive prediction was a methylated fragment and a negative prediction was unmethylated. The fragments' best (maximum of the 5) F1 and MCC score was used as its predicted residue.

The methylation fragments used are as follows, where x is a methylated cytosine.

In this theoretical example, reads compared against a 45% oligo residue, false Positive Methylated Cytosine bases are represented by X and False Negative cytosine are indicated by C:

(SEQ ID NO: 8)
tcgatxgttcgaccgactcggCgataatgttCgxgaxgctxgctxtgaaa
ttcgCgaacgcxacgcxtxgagtgaagaxgxgattgxgcacxatcgcgct
gxgccgtcaxgcgxgtagaaactxgtcgcgagaxgcgxagxgggxggaxg
xgatxagxxtgagatattxgtgttgtgxgtxgtxgcgxgxgcagtcaggc

Comparing it to the 45% oligonucleotide fragment would yield:

| Fragment Example Score Matrix, | | |
|---|---|---|
| 45% methylated oligonucleotide | | |
| (64 bases in length) | 45% residue M | 45% Residue C |
| Residue called M | 26 | 5 |
| Residue called C | 3 | 30 |

To reiterate, this confusion matrix was then used to calculate MCC and F1. This was done five times for each read*potential oligo. If the maxima of these F1 and MCC agreed on the call of the fragment, then it was determined to be that fragment.

This method was extended to look at the ideal base calling quality score cutoff to use called methylated cytosine >X, where X was between 0-255 and the mean MCC/F1 for all fragments at that cutoff. The highest scoring cutoff was then chosen for additional by base residue analysis.

For the by-residue analysis or per residue, the fragments were separated into their max MCC and F1 typed 5%-90% types. Each bases' accuracy was examined, where accuracy is simply the number of times the fragment was called correctly over the total depth.

Fingerprint barcodes also were examined within our fragments. Unfortunately, depth of the forward fragments fell off toward the tail end, so these were not always present in most of the fragments and could not be relied upon to correctly identify each read.

### Analysis

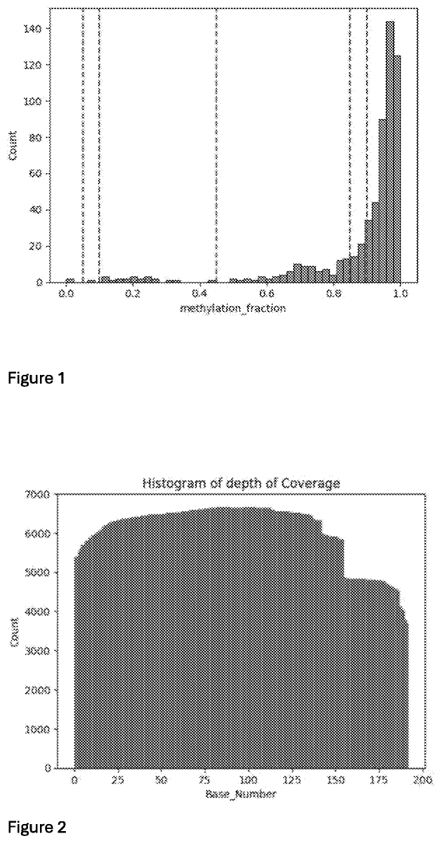

The initial analysis of the oligonucleotides discussed above demonstrated that amplification of the oligonucleotides was achieved with the oligonucleotides most amplified being highly methylated. This was done at a cutoff of 150 with barcode 1 of 8 and Flowcell 1 of 2. See FIG. 1. The bars are the fragments' methylation score bins corresponding to the level of methylation.

Looking at the counts per bin using all barcodes and the method of determination (max MCC & F1) described in the methods section, similar findings were achieved for both flowcells and all barcodes concatenated together.

TABLE 2
Flowcell 1 (@ Quality cutoff 25)

| Percent methylation | Count |
|---|---|
| 5% | 9 |
| 10% | 16 |
| 45% | 416 |
| 85% | 3479 |
| 80% | 6 |

TABLE 3
Flowell 2 (@ Quality cutoff 7)

| Percent methylation | Count |
|---|---|
| 5% | 10 |
| 10% | 9 |
| 45% | 373 |
| 85% | 6347 |
| 80% | 18 |

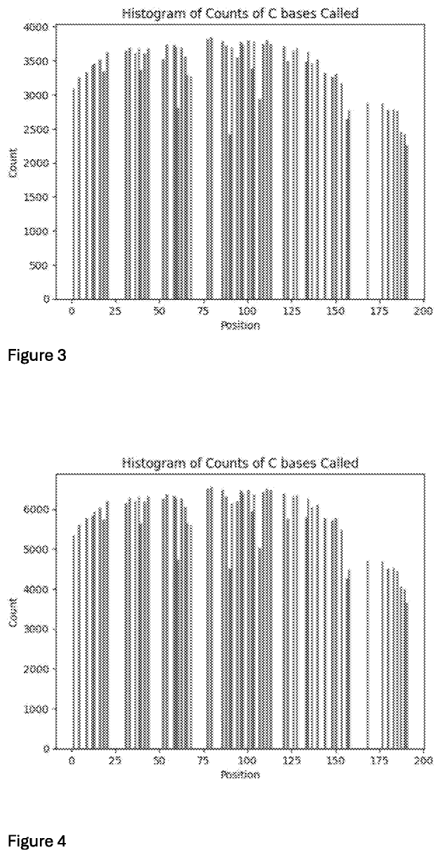

For example, looking at the depth of each cytosine base, both Flowcell 1 (FIG. 3) and Flowcell 2 (FIG. 4) respectively have bases that are missing from the final fragments. Also, the insertion and deletion errors were quite high on these residues. See FIGS. 3 and 4.

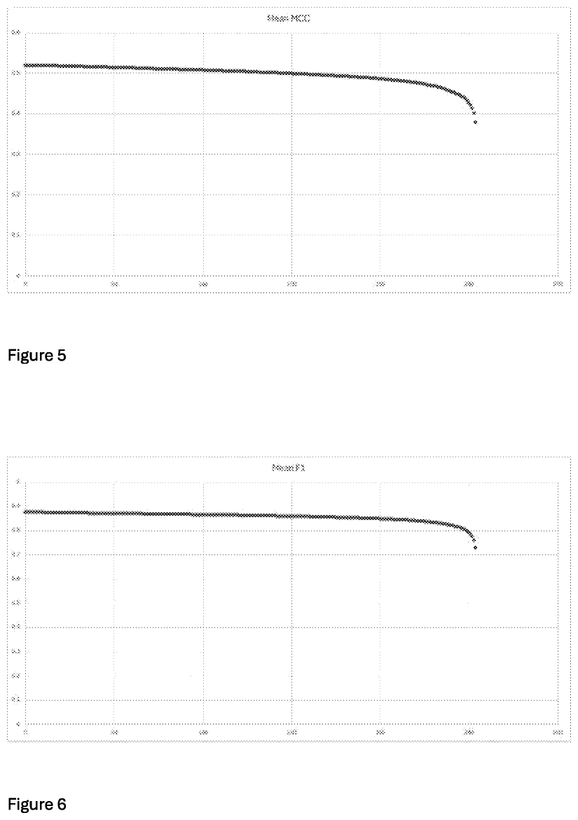

This lead to characterizing F1 and MCC mean/MCC max curves (FIGS. 5 and 6) at all quality scores when varying the call M at >quality bar for both flowcells.

As noted in Tables 2 and 3, the majority of the reads were classified as 85% methylated oligonucleotide reads. Without wishing to be bound by any particular theory, the difference between 90% and 85% type reads may have been low. The error rate on calling a methylated fragment correctly was such that the majority of the reads were binned in the 85% bin, as the accuracy loss by miss-calling overall was less than the accuracy loss of the few select bases. As shown in FIG. 2, the select loss of later methylated oligonucleotides meant that more oligonucleotides were classified as 85% methylated as the differences between 85% methylated and 90% methylated were tied to the later end of the read.

The curves in FIGS. 5 and 6 show that there was selective amplification/isolation of methylated residues, as the cost of calling a base non-methylated was greater than the cost of calling it methylated. This is seen in the drop off to the right of each graph; while the left side shows a drop off, it does not show as significant of a curve. These curves show that the ideal level setting methylation calling score for all reads on the flowcell. In this instance, the maximum mean value of MCC of 25 was chosen on flowcell 1 and 7 on flowcell 2. Each flow cells' base calls at each maxima was examined.

Here, particular bases are not captured well and are routinely called incorrectly. There were no bases called C in the 90% fragment but called methylated in the 85% fragment. This meant that 90% fragment identity was washed out by the basal false positive rate of calling all fragments. The difference in methylated fragments in our 85% and 90% calls were identified by their bad performance, also the poor performance of bases which were methylated in none of the fragments. Changes between bases at positions 44 and 66 in the 90% fragment, for example, were challenged by the true C base of position 13, which also shared a bad performance on F1 and MCC and its need to remain C. This difference resulted both in our Quality score optimization result and in our high call of 85% fragments. This suggests an advantage in switching methylated bases in higher and lower percentage fragments in identical sequence strands. When an oligonucleotide fragment is methylated in one fraction the population of oligonucleotides and an oligonucleotide fragment is similarly methylated in another fraction of the population, this reduces the likelihood that the F1 score or MCC score will correctly classify the molecule by methylation fingerprint. This is also observed when true positive and true negatives overlap too much, which reduces the effectiveness of false calls in either direction. As such, there may be an advantage in shifting the position of a methylated base within fractions in a population while keeping the overall level of methylation constant.

1. A composition comprising four or more unique oligonucleotides,
each of the four or more oligonucleotides comprising a unique nucleotide sequence and a unique number of methylated bases,
wherein the unique number of methylated bases is about 0% to 100% of the unique nucleotide sequence of each of the four or more oligonucleotides.

2. The composition of claim 1, wherein each of the oligonucleotides comprises one or more methylation motifs.

3. The composition of claim 1, wherein the oligonucleotide comprises a hairpin.

4. The composition of claim 1, wherein the oligonucleotide comprises a barcode adjacent to the hairpin.

5. The composition of claim 1, wherein each of the oligonucleotides comprises multiple methylation motifs and each methylation motif has a unique percentage of methylation.

6. The composition of claim 1, wherein one or more of the oligonucleotides comprise one or more methylation motifs comprising any one of SEQ ID NOs: 1-14.

7. The composition of claim 1, wherein at least one oligonucleotide comprises single-stranded RNA.

8. The composition of claim 1, wherein at least one oligonucleotide comprises double-stranded RNA.

9. The composition of claim 1, wherein the oligonucleotides in the composition are present in different concentrations.

10. The composition of claim 1, comprising at least ten unique oligonucleotides.

11. The composition of claim 1, comprising a first oligonucleotide comprising a nucleic acid sequence that is 0.1% methylated, a second oligonucleotide comprising a nucleic acid sequence that is 0.5% methylated, a third oligonucleotide comprising a nucleic acid sequence that is 16% methylated, a fourth oligonucleotide comprising a nucleic acid sequence that is 40% methylated, and a fifth oligonucleotide comprising a nucleic acid that is 80% methylated.

12. The composition of claim 1, wherein the oligonucleotides have a methylation profile along a linear, logarithmic, exponential, semi-log scale, and/or sigmoidal curve.

13. A composition comprising four or more oligonucleotides each having a unique pattern of methylated bases in methylation motifs, wherein the each of the unique oligonucleotides have a percent of methylated bases between 0.1% and 100%.

14. The composition of claim 1, wherein the oligonucleotides are 100 nucleotides to 400 nucleotides in length.

15. (canceled)

16. The composition of claim 1, wherein one or more of the methylated bases are N6-Methyladenine, 5-Methylcytosine, or 5-HydroxyMethylcytosine.

17. The composition of claim 1, comprising an oligonucleotide comprising a nucleic acid sequence having 0.5% methylated bases.

18. The composition of claim 1, wherein each of the oligonucleotides share less than 25% sequence identity with a human gene.

19. A method, comprising:
a) adding a known amount of the composition of claim 1 having a predetermined percent methylation to a test sample comprising a target nucleic acid;
b) determining the percent methylation of the target nucleic acid; and
c) determining the percent methylation of oligonucleotides in the composition of claim .

20. A method comprising:
a) adding a known amount of a composition of claim 1 having a predetermined percent methylation to a test sample;
b) identifying the percent methylation of each of the oligonucleotides in the test sample; and;
c) determining a percent methylation cutoff value based on step (b).

21. The method of claim 19, wherein the percent methylation is determined by 5hmC sequencing, CpG sites sequencing (MCTA-seq), methylated DNA immunoprecipitation (cfMeDIP-seq), methylation ddPCR (qMSP), methylation detection using ionic current signal from Nanopore sequencing, targeted bisulfite sequencing, TET-assisted pyridine borane sequencing (TAPS), whole genome bisulfite sequencing (WGBS), or any combination thereof.

22-25. (canceled)
